(19) United States
(12) Patent Application Publication
(10) Pub. No.: US 2006/0078983 A1
(43) Pub. Date: Apr. 13, 2006

(54) Polymer monolith substrate

(75) Inventor: Aldrich N.K. Lau, Palo Alto, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 11/213,635

(22) Filed: Aug. 25, 2005

Related U.S. Application Data
(63) Continuation-in-part of application No. 60/604,927, filed on Aug. 27, 2004.

(51) Int. Cl. C12M 1/34; C08L 43/00

(52) U.S. Cl. 435/287.1; 525/209

(57) ABSTRACT

The present teachings provide for composite substrates for the covalent attachment of biomolecules and method of making the same. The present teachings provide for composite substrates comprising a porous copolymer-monolith covalently attached to a surface of a substrate, wherein the porous copolymer-monolith has been formed by an inverse phase photo-copolymerization process comprising photo-copolymerizing at least one ethylenically unsaturated monomer with polymerizable surface functionalities that are covalently attached to a surface of a derivitized substrate such that, after photo-copolymerization, the porous copolymer-monolith is covalently attached to the surface of the substrate, and wherein the photo-copolymerizing is carried out in the presence of at least one porogenic solvent.

This application claims a priority benefit under 35 U.S.C. § 119(e) from U.S. Patent Application No. 60/604,927, filed Aug. 27, 2004, which is incorporated herein by reference

The present teachings generally relate to solid supports for the immobilization of biomolecules.

The detection of nucleic acids in a biological sample has become an important application in, among others, the areas of the medicine, forensics, agriculture, and food science. Various methods in a variety of assay formats have been advanced for detecting nucleic acids. Among the most popular methods is the hybridization of a labeled polynucleotide to a complimentary polynucleotide that has been attached to a solid support. Numerous solid supports have been used for the immobilization of polynucleotides including, but not limited to, nitrocellulose, activated agarose, glass, polymers, for example polystyrene and nylon, and various polymer-coated surfaces. Furthermore, these solid supports have been developed in a variety of formats including, membranes, microtiter plates, beads, particles, arrays, and the like. For example, microarrays have rapidly developed into powerful and highly sensitive tools for use in, for example, the medical, forensics and biological sciences.

In microarray technology, the covalent immobilization of polynucleotides is usually achieved in one of two ways. In one approach, polynucleotide targets are synthesized directly on a solid support. See, for example, Fodor, et al., U.S. Pat. No. 5,424,186; Pirrung, et al., U.S. Pat. No. 5,143,854 and Bass, et al. U.S. Pat. No. 6,440,669. In an alternative approach, referred to herein as the spotting method (or delivery method), polynucleotides are synthesized prior to immobilization and then coupled to a solid support. See, for example, Okamoto, T., et al., U.S. Pat. No. 6,476,215; Bruhn, et al., U.S. Pat. No. 6,458,853 and Southern, E., U.S. Pat. No. 5,700,637. In the spotting method, spotting is generally achieved by reaction of a nucleophilic group on a surface of a solid support with a reactive group on a polynucleotide that is capable of reacting with the nucleophilic group on the solid support to form a covalent bond, or alternatively, a surface of a solid support can be functionalized to present a reactive group that is capable of reacting with a nucleophile on the 3′- or 5′-end of a polynucleotide.

It is generally known in the art that a microarray substrate should ideally possess several basic characteristics. For example, be able to withstand the conditions under which biomolecules will be attached (i.e.—by covalent attachment or passive adsorption) and any analytical methods carried out. For example, for hybridization assays of polynucleotides in genetic analysis, the microarray substrate must be able to withstand hybridization and washing conditions that can often include prolonged exposure to aqueous buffers at elevated temperatures.

In addition, a microarray substrate must provide a means by which a biomolecule of interest (i.e.—polynucleotide probes) can be attached to the surface. Typically there are two general ways in which biomolecules, for example are attached to the surface of a microarray substrate. First, polynucleotides can be attached by non-covalent passive adsorption onto a charged surface of a microarray substrate. This is typically accomplished by providing a charged surface, such as an amine derivitized surface, and contacting the surface with a plurality of polynucleotides under conditions suitable to provide non-covalent absorption of the polynucleotides onto the amine surface. Alternatively, biomolecules of interest can be covalently attached to the surface of the microarray substrate. Because of the robust nature of the attachment and the increased density of biomolecules within a given feature that covalent attachment can provide, this has become the preferred method of attachment of biomolecules in the microarray field. To achieve the covalent attachment of biomolecule targets (i.e.—polynucleotides) on the surface of the microarray substrate, the substrate surface must contain some functional group that is capable of reacting with a complimentary functional group on the biomolecule to form a stable covalent bond. As a result potential microarray substrates should be designed to be amenable to further surface chemistries.

Accordingly, there exists a need to provide an economical microarray substrate that can be used in a variety of microarray applications.

In some embodiments, the present teachings can provide a composite substrate comprising a porous copolymer-monolith covalently attached to a surface of a substrate, wherein the porous copolymer-monolith has been formed by an inverse phase photo-copolymerization process comprising photo-copolymerizing at least one ethylenically unsaturated monomer with polymerizable surface functionalities that are covalently attached to a surface of a derivitized substrate such that, after photo-copolymerization, the porous copolymer-monolith is covalently attached to the surface of the substrate, and wherein the photo-copolymerizing is carried out in the presence of at least one porogenic solvent.

In some embodiments, the substrate can be a polymer or glass. In some embodiments, the polymerizable surface functionalities can be acrylates, methacrylates, acrylamides, methacrylamides, vinylic moieties, allylic moieties, and combinations thereof. In some embodiments, the substrate can be glass. In some embodiments, the derivitized substrate comprises a substrate and at least one attaching moiety containing polymerizable surface functionalities covalently attached to the substrate. In some embodiments, the attaching moiety can be a silane. In some embodiments, the silane can be 3-(trimethoxysilyl)propyl (meth)acrylate, 3-(triethoxysilyl)propyl (meth)acrylate, 3-(dimethoxymethylsilyl)propyl (meth)acrylate, 3-(diethoxymethylsilyl)propyl (meth)-acrylate, 3-(methoxydimethylsilyl)propyl (meth)acrylate, 3-(ethoxydimethylsilyl)propyl (meth)acrylate and combinations thereof.

In some embodiments, the inverse phase photo-copolymerization further comprises at least one ethylenically unsaturated cross-linker that contains two or more ethylenically unsaturated moieties. In some embodiments, the at least one ethylenically unsaturated monomer comprises acrylic acid. In some embodiments, the at least one ethylenically unsaturated monomer comprises acrylic acid and at least one other ethylenically unsaturated monomer.

In some embodiments, the at least one ethylenically unsaturated monomer can be acrylic acid, methyl acrylate, butyl acrylate, or any combination thereof. In some embodiments, the ethylenically unsaturated cross-linkers can be N,N-methylenebisacrylamide, ethylene glycol di(meth)acrylate, propylene glycol di(meth)acrylate, poly(ethylene glycol) diacrylate, or any combination thereof. In some embodiments, the at least one ethylenically unsaturated monomer can be acrylic acid and the at least one ethylenically unsaturated cross-linker can be N,N-methylenebisacrylamide. In some embodiments, the at least one ethylenically unsaturated monomer can be acrylic acid and methyl methacrylate and the at least one ethylenically unsaturated cross-linker can be N,N-methylenebisacrylamide. In some embodiments, the at least one ethylenically unsaturated monomer can be acrylic acid and butyl acrylate and the at least one ethylenically unsaturated cross-linker can be ethylene glycol diacrylate. In some embodiments, the at least one ethylenically unsaturated monomer can be acrylic acid and butyl acrylate and the at least one ethylenically unsaturated cross-linker can be poly(ethylene glycol) diacrylate. In some embodiments, the at least one ethylenically unsaturated monomer can be acrylic acid and methyl acrylate and the at least one ethylenically unsaturated cross-linker can be poly(ethylene glycol) diacrylate.

In some embodiments, prior to polymerization, the at least one ethylenically unsaturated monomer comprises from about 10 to about 30 wt % of acrylic acid. In some embodiments, prior to polymerization, the at least one ethylenically unsaturated monomer comprises from about 40 to about 98 wt % of acrylic acid. In some embodiments, prior to polymerization, the at least one ethylenically unsaturated monomer comprises from about 60 to about 90 wt % of acrylic acid. In some embodiments, prior to polymerization, the at least one ethylenically unsaturated monomer comprises from about 30 to about 60 wt % of butyl acrylate. In some embodiments, prior to polymerization, the at least one ethylenically unsaturated monomer comprises from about 50 to about 60 wt % of butyl acrylate. In some embodiments, prior to polymerization, the at least one ethylenically unsaturated monomer comprises from about 10 to about 25 wt % of methyl methacrylate. In some embodiments, prior to polymerization, the photo-copolymerization comprises from about 1 to about 50 wt % of at least one ethylenically unsaturated cross-linker. In some embodiments, prior to polymerization, the photo-copolymerization comprises from about 5 to about 30 wt % of at least one ethylenically unsaturated cross-linker. In some embodiments, prior to polymerization, the photo-copolymerization comprises from about 10 to about 30 wt % of at least one ethylenically unsaturated cross-linker. In some embodiments, prior to polymerization, the photo-copolymerization comprises from about 10 to about 20 wt % of at least one ethylenically unsaturated cross-linker.

In some embodiments, the present teachings provide for a composite substrate further comprising at least one non-reflective additive intercalated within the porous copolymer-monolith. In some embodiments, the non-reflective additive can be carbon black.

In some embodiments, the present teachings provide for methods of fabricating a composite substrate comprising:

i) contacting a derivitized substrate having polymerizable surface functionalities covalently attached thereto with a solution comprising at least one porogenic solvent, at least one photopolymerization initiator, and at least one ethylenically unsaturated monomer; and

ii) copolymerizing the polymerizable surface functionalities with the at least one ethylenically unsaturated monomer to form a porous-copolymer monolith covalently attached to a substrate. In some embodiments, the solution further comprises at least one ethylenically unsaturated cross-linker.

In some embodiments, the photoinitiator can be a unimolecular initiator, a bimolecular initiator, or combinations thereof. In some embodiments, the photoinitiator comprises benzophenone and methyl 3-(dimethylamino)benzoate. In some embodiments, the photoinitiator comprises benzophenone and ethyl 4-(dimethyl-amino)benzoate. In some embodiments, the at least one porogenic solvent can be pentadecane, 2-butanone, dioxane, heptane, ethyl ether, or any combination thereof. In some embodiments, the at least one porogenic solvent can be pentadecane. In some embodiments, the at least one porogenic solvent can be 2-butanone.

It is to be understood that both the foregoing general description and the following detailed description are exemplary and explanatory only and are not restrictive of the present teachings. In this application, the use of the singular includes the plural unless specifically stated otherwise. In this application, the use of “or” means “and/or” unless stated otherwise. Furthermore, the use of the term “including,” as well as other forms, such as “includes” and “included,” is not limiting.

It will be understood that composite substrates as defined herein can serve as array or microarray substrates. As used herein, “array” refers to a positionally addressable arrangement of targets or polymers (i.e.—polynucleotides) on a solid support, wherein the solid support comprises, for example, a planar substrate, and each target is located at a known, predetermined location on the solid support such that the identity of each target can be determined from its location on the solid support. Each “location” having a target attached thereto will be referred to herein as a “feature”. As used herein “array” refers to positionally addressable arrangement of targets or polymers having a density of less than about 150 features per 1 cm², wherein each feature can have attached thereto a different target.

As used herein “microarray” refers to arrays having a density of at least 150 features per 1 cm² or greater (150 features/cm²), wherein each feature can have attached thereto a different target. In some embodiments, the density of features is at least 250 features per 1 cm² or greater. In some embodiments, the density of features is at least 500 features per 1 cm² or greater. In some embodiments, the density of features is at least 1000 features per 1 cm² or greater. In some embodiments, the density of features is is at least 1250 features per 1 cm² or greater. In some embodiments, the density of features is at least 1500 features per 1 cm² or greater. In some embodiments, the density of features is at least 2000 features per 1 cm² or greater. In some embodiments, the density of features is at least 2500 features per 1 cm² or greater.

In some embodiments, the density of features is in a range from 250 to about 1000 features per 1 cm². In some embodiments, the density of features is in a range from 1000 to about 5000 features per 1 cm². In some embodiments, the density of features is in a range from 5000 to about 10000 features per 1 cm². In some embodiments, the density of features is in a range from 10000 to about 15000 features per 1 cm². In some embodiments, the density of features is in a range from 15000 to about 20000 features per 1 cm².

In some embodiments, arrays and microarrays can comprise a plurality of beads in a positionally addressable arrangement. Such arrays and microarrays known as “bead arrays” or “bead microarrays” are known in the art. See, for example, Chee, M. S., et al., U.S. Pat. No. 6,429,027, Stuelpnagel, J. R., et al., U.S. Pat. No. 6,396,995, Chee, M. S., et al., U.S. Pat. No. 6,355,431 and references cited therein. In some embodiments, the present teachings provide bead arrays comprising a plurality of positionally addressable beads wherein at least one bead comprises a porous copolymer-monolith covalently attached thereto, wherein the porous copolymer-monolith has been formed by an inverse phase photo-copolymerization process comprising photo-copolymerizing at least one ethylenically unsaturated monomer with polymerizable surface functionalities that are covalently attached to a surface of a derivitized bead such that, after photo-copolymerization, the porous copolymer-monolith is covalently attached to the surface of the bead, and wherein the photo-copolymerizing is carried out in the presence of at least one porogenic solvent.

Suitable substrates for use in connection with the present teachings can be of a variety of materials and configurations. Among others, suitable substrate materials include but are not limited to organic and inorganic substrates, and the like. Inorganic substrates can include, but are not limited to, metals, semi-conductor materials, glasses and ceramics. Examples of metals that can be used as substrate materials include, but are not limited to, gold, platinum, nickel, palladium, aluminum, steel, chromium and gallium arsenide. Semiconductor materials that can be used as substrate materials include silicon and germanium. Glass and ceramic materials that can be used as substrate materials include, but are not limited to, commercial glasses, such as those made of a composition that comprises sand and soda ash (i.e.—soda-lime glass), lead glasses of the type that comprise lead oxide additives, borosilicate glasses such as those which comprise silica and borosilicate and may include additional additives (i.e.—Pyrex glass and alkaline earth aluminoborosilicate), vitreous silica, aluminosilicate glass of the type that comprises aluminum oxide and may contain additional additives, alkalibariumsilicate glass, borate glass, phosphate glass, chalcogenide glass, quartz glass, porcelain and further metal oxides which are understood to mean ceramic materials. Further examples of inorganic substrates include but are not limited to graphite, zinc selenide, mica, silicon dioxide, lithium niobate and further supports.

Organic substrates for use in connection with the present teachings include but are not limited to polymeric materials such as polyesters (i.e.—polyethylene terephthalate, polybutylene terephthalate, and the like), polyvinyl chloride, polyvinylidene fluoride, polyvinylidenedifluoride, polytetrafluoroethylene (PTFE), polycarbonate, polyamide, poly(methyl(meth)acrylate), polystyrene, poly(alkylolefins), such as polyethylene and polypropylene, poly(cyclic olefins), poly(vinyl acetate), epoxy resins, polyurethanes, cellulose, cellulose esters, and the like, and combinations thereof (i.e.—copolymers), wherein any polymer can be modified to include charged, polar, hydrophilic, nucleophilic and/or electrophilic groups. Copolymers for use in the present teachings include copolymer blends of polymers, such as those listed above, and copolymers of more than one monomer type (i.e.—random copolymers, pseudo-copolymers, statistical copolymers, statistical pseudo-copolymers, alternating copolymers, periodic copolymers and block copolymers as defined by IUPAC in Glossary of Basic Terms in Polymer Science, (IUPAC Recommendations 1996) Eds. Jenkins, A. D., Kratochvil P., Stepto R. F. T. and Suter U. W.

It will be understood that substrates for use in connection with the present teachings include, but are not limited to, beads, membranes, resins, particles, granules, gels and planar substrates (i.e.—glass or plastic slides). Substrates for use in connection with the present teachings can be porous or non-porous. Substrates for use in connection with the present teachings can be planar, substantially planar or non-planar. Furthermore, substrates for use in connection with the present teachings can be freestanding (i.e.—where a porous copolymer-monolith of the present teachings is attached directly or through an attaching moiety to a substrate) or part of a composite substrate (e.g.—where a porous copolymer-monolith of the present teachings is attached directly or through an attaching moiety a polymeric membrane, such as nylon, that is in turn attached to a planar substrate).

In some embodiments, the substrate comprises glass. In some embodiments, the substrate comprises a glass slide. In some embodiments, the substrate comprises a Pyrex slide. In some embodiment the substrate comprises a glass wafer. In some embodiments, the substrate comprises tinted glass. In some embodiments, the substrate comprises black glass. In some embodiments, the substrate comprises a PTFE block. In some embodiments, the substrate comprises a PTFE wafer.

As used herein, the term “inverse phase photo-polymerization” or “inverse phase photo-copolymerization”, which are used interchangeably unless otherwise specified, means a polymerization process wherein at least one photopolymerizable monomer is polymerized in an organic porogen or a mixture of organic porogens under conditions such that as polymerization or copolymerization proceeds, the polymer or copolymer that is formed becomes the continuous phase and the porogen or mixture of porogens becomes the discrete phase. It will be understood by one of skill in the art that by such a process, it is possible to form a porous-polymer monolithic structure or porous-copolymer monolithic structure.

The “inverse phase photo-polymerization” process used in connection with the present teachings can be contrasted to standard emulsion polymerization. Specifically, in standard emulsion polymerization, the solvent, usually an aqueous solvent, is the continuous phase throughout the polymerization and the polymer particles formed during polymerization are the discrete phase. It will also be understood by those skilled in the art that standard inverse emulsion polymerization can give rise to water soluble polymer microspheres or in the presence of a cross-linker, can give rise to discrete microspheres of a water-swellable hydrogel. Specifically, it is known in the art that water-swellable hydrogels in the form of a surface coating can be produced by in situ emulsion polymerization on a substrate surface in the presence of a thermal initiator (see, for example, Sundberg, et al. U.S. Pat. No. 5,624,711). One of skill in the art will recognize that by using an “inverse phase photo-polymerization”, the present teachings can provide for a porous-copolymer monolith for application in microarray applications (i.e.—that is resistance to swelling in the presence of aqueous buffer).

Accordingly, composite substrates of the present teachings can be formed by an inverse phase photo-copolymerization process in which polymerizable surface functionalities that are covalently attached to a derivitized substrate are copolymerized together with at least one ethylenically unsaturated monomer in the presence of at least one porogenic solvent under conditions capable of forming a porous copolymer-monolith covalently attached to a surface of a substrate.

In some embodiments, the present teachings provide for methods of fabricating a composite substrate comprising:

i) contacting a derivitized substrate having polymerizable surface functionalities covalently attached thereto with a solution comprising at least one porogenic solvent, at least one photopolymerization initiator, and at least one ethylenically unsaturated monomer; and

ii) copolymerizing the polymerizable surface functionalities with the at least one ethylenically unsaturated monomer to form a porous-copolymer monolith covalently attached to a substrate. In some embodiments, the solution further comprises at least one ethylenically unsaturated cross-linker.

As used herein, the term “polymerizable surface functionalities” refers to any functionality comprising an ethylene moiety that is covalently attached itself, or through an attaching moiety, to a substrate. It will be understood by those of skill in the art that the nature of the attaching moiety by which the “polymerizable surface functionalities” is capable of covalently attaching to a surface of a substrate will vary depending on the nature of the substrate material. For example, when the substrate is composed of a glass surface or bead, suitable attaching moieties can be those comprising at least one silane functionality and at least one ethylenically unsaturated moiety (i.e.—the polymerizable functionality). In such an example, silanol groups on the surface of the glass can react with the silane functionality (i.e.—an alkoxysilane moiety) of an organo-silane to form a silicon-oxygen covalent bond. Suitable organo-silanes include those comprising monoalkoxysilanes, dialkoxysilanes and trialkoxysilanes. In some embodiments, suitable alkoxysilanes for use in connection with the present teachings when the substrate comprises a glass substrate or bead include, but are not limited to acrylamide, acrylate, methacrylamide and methacrylate derivatives of hydroxyl functionalized silanes, such as mono-, di- and tri-alkoxy hydroxyalkylsilanes and amine functionalized silanes, such as mono-, di- and tri-alkoxy aminoalkylsilanes.

Examples of suitable alkoxysilanes for use in connection with the present teachings include, but are not limited to 3-(tris(trimethylsiloxy)silyl)propyl (meth)acrylate, 3-(tris(trimethylsiloxy)silyl)propyl (meth)acrylamide, N-[N′-(3-(tri-methoxysilyl)propyl)-2-aminoethyl]-2-aminoethyl (meth)acrylamide, N-[N′-(3-(tri-methoxysilyl)propyl)-2-aminoethyl]-2-aminoethyl (meth)acrylate, N-[3-((dimethoxy)-methylsilyl)propyl]-2-aminoethyl (meth)acrylamide, N-[3-((dimethoxy)methyl-silyl)-propyl]-2-aminoethyl (meth)acrylate, N-[3-(trimethoxysilyl)propyl]-2-aminoethyl (meth)acrylamide, N-[3-(trimethoxysilyl)propyl]-2-amino-ethyl (meth)acrylate, 3-(trimethoxysilyl)propyl (meth)acrylamide, 3-(trimethoxysilyl)propyl (meth)acrylate, N-methyl-(N-3-(tri-methoxysilyl)propyl) (meth)acrylamide, N-phenyl-(N-3-(trimethoxy-silyl)propyl) (meth)acrylamide, 3-(triethoxysilyl)propyl (meth)acrylamide, 3-(triethoxy-silyl)propyl (meth)acrylate, trimethoxy(vinyl)silane, allyltriethoxysilane, allyltrimethoxy-silane, allyldimethoxymethylsilane, allyldiethoxymethylsilane, 3-(N-allylamino)propyl-trimethoxysilane, allyltri(trimethylsilyloxy)silane, 3-((diethoxy)methylsilyl)propyl (meth)acrylamide, 3-((diethoxy)methylsilyl)propyl (meth)acrylate, 3-(triethoxysilyl)-propyl (meth)acrylamide, 3-(triethoxysilyl)propyl (meth)acrylate, 3-(tris-[2-(2-methoxy-ethoxy)ethoxy]silyl)propyl (meth)acrylamide, 3-(tris-[2-(2-methoxyethoxy)ethoxy]-silyl)propyl (meth)acrylate, N,N-[bis(2-(meth)acryloxyethyl)]-3-aminopropyl-(triethoxy)-silane, 3-(diethoxymethylsilyl)propyl (meth)acrylate, 3-(diethoxymethylsilyl)propyl (meth)acrylamide, diethoxy(methyl)vinylsilane, ethoxy(dimethyl)vinylsilane, triethoxy-(vinyl)silane, trimethoxy(7-octen-1-yl)silane, 3-[tris(2-methoxyethoxy)silyl]propyl (meth)acrylamide, 3-[tris(2-methoxyethoxy)silyl]propyl (meth)acrylate, tris(2-methoxy-ethoxy)vinylsilane, N,N-[bis(2-(meth)acryloxyethyl)-3-aminopropyl-(methyldiethoxy)-silane, 3-(N-methyl-N-(meth)acrylamino)propyl(methyldimethoxy)silane, N-((meth)acryloxyethyl)-N-methylaminopropyl(trimethoxy)silane, and the like. It will be understood by those of skill in that art that as used herein, “(meth)acrylate”, “(meth)acrylamide” and the like encompass both the methylated and unmethylated forms in accordance with the ordinary nomenclature used by those of skill in the art. For example, 3-(trimethoxysilyl)propyl (meth)acrylate refers to and provides support for both 3-(trimethoxysilyl)propyl acrylate and 3-(trimethoxysilyl)propyl methacrylate. It will also be understood by those of skill in the art that numerous other combinations of substrate materials and attaching moieties comprising polymerizable functionalities are possible.

In some embodiments, the alkoxysilane can be one or more of 3-(trimethoxysilyl)propyl (meth)acrylate, 3-(triethoxysilyl)propyl (meth)acrylate, 3-(tributoxysilyl)propyl (meth)acrylate, 3-(triisopropoxysilyl)propyl (meth)acrylate, 3-(dimethoxymethylsilyl)propyl (meth)acrylate, 3-(diethoxymethylsilyl)propyl (meth)acrylate, 3-(dibutoxymethylsilyl)propyl (meth)acrylate, 3-(diisopropoxymethyl-silyl)propyl (meth)acrylate, 3-(methoxydimethyl)propyl (meth)acrylate, 3-(ethoxy-dimethyl)propyl (meth)acrylate, 3-(isopropoxydimethyl)propyl (meth)acrylate and 3-(butoxydimethyl)propyl (meth)acrylate.

In some embodiments, the alkoxysilane can be one or more of 3-(trimethoxysilyl)propyl (meth)acrylate, 3-(triethoxysilyl)propyl (meth)acrylate, 3-(dimethoxymethylsilyl)propyl (meth)acrylate, 3-(diethoxymethylsilyl)propyl (meth)acrylate, 3-(methoxydimethyl)propyl (meth)acrylate and 3-(ethoxydimethyl)propyl (meth)acrylate.

As used herein, the term “ethylenically unsaturated monomer” refers to any monomer comprising a polymerizable ethylenically unsaturated moiety. Suitable ethylenically unsaturated monomers for use in connection with the present teachings include, but are not limited to, vinylic monomers, allylic monomers, acrylate monomers, acrylamide monomers, acrylic acid monomers, and the like. Furthermore, ethylenically unsaturated monomers can optionally contain additional reactive moieties that may not be directly involved in the polymerization process, but can optionally be present on the porous copolymer surface for further reactions with polymeric biomolecules (e.g.—proteins, polynucleotides, peptide nucleic acids (PNAs), locked nucleic acids (LNAs), PNA-DNA chimeras, and the like), amino acid monomers, nucleotide monomers, small molecules, other polymers (e.g.—nylon and other membranes), and the like.

It will be understood that the combination of ethylenically unsaturated monomers used to make a porous monolith copolymer in connection with the present teachings will depend on what reactive moiety or functional group is to be presented on the porous copolymer-monolith surface. Suitable reactive moiety or functional groups include, but are not limited to, carboxylic acids, sulfonic acids, amines, alcohols, isocyanates, isothiocyanates, thiols, selenides, epoxides, and the like. Accordingly, suitable ethylenically unsaturated monomers include, but are not limited to, those that, after polymerization provide a porous copolymer-monolith having on its surface at least one reactive moiety selected from carboxylic acids, succinimide, sulfonic acids, aldehydes, amines, alcohols, isocyanates, isothiocyanates, thiols, selenides, epoxides, azolactone, and the like.

It will be understood that the desired reactive moiety to be presented on porous copolymer-monolith surface will depend on the specific application of the substrate being formed. For example, methods of synthesizing arrays of biopolymers, including oligonucleotides, peptides and other polymers have been described previously (see, for example, Pirrung, et al., U.S. Pat. No. 5,143,854, Fodor, et al., PCT Publication No. WO 92/10092, and Fodor, et al., Science, 251:767-777 (1991), each of which is incorporated herein by reference for all it discloses). In the methods described in the above publications, reactive moieties, such as amino groups, hydroxyl groups and isothiocyanate groups can be used to provide an attachment site at which a biopolymer can be synthesized according to the methods described by each publication. One of skill in the art will recognize that a variety of reactive moieties and functional groups can be included on the surface of a porous polymer as a site for beginning synthesis of a biopolymer, and that it is often necessary to derivatize, modify or in some way alter the reactive moiety or functional group prior to beginning biopolymer synthesis.

Alternatively, a variety of methods for covalently attaching pre-synthesized biopolymers to a solid support surface have been disclosed. Examples of such methods include, but are not limited to, the reaction of a sulfonyl chloride attached to a surface with an amine reactive group on the 3′ or 5′-end of and oligonucleotide, the reaction of an N-hydroxysuccinimidyl (NHS) carbamate with an amine reactive group on the 3′ or 5′-end of and oligonucleotide, reaction of an activated ester, such as an NHS ester, with an amine reactive group on the 3′ or 5′-end of and oligonucleotide, reaction of an isocyanate with an amine reactive group on the 3′ or 5′-end of and oligonucleotide or an amine reactive group on a peptide. Accordingly, the reactive moieties or functional groups presented on the surface of a porous copolymer-monolith for these methods can include amines, carboxylic acids, sulfonic acids, isocyanates, and the like.

Suitable ethylenically unsaturated monomers for use in connection with the present teachings include, but are not limited to, those of the formula I:

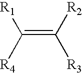

where R₁, R₂ and R₃ can each independently be, for example, hydrogen, halogen, C₁-C₁₂ unsubstituted linear alkyl, C₃-C₁₂ unsubstituted cyclic alkyl, C₃-C₁₂ unsubstituted branched alkyl, C₆-C₂₀ unsubstituted aryl, C₆-C₂₀ unsubstituted heteroaryl, C₁-C₁₂ substituted linear alkyl, C₃-C₁₂ substituted cyclic alkyl, C₃-C₁₂ substituted branched alkyl, C₆-C₂₀ unsubstituted aryl and C₆-C₂₀ unsubstituted heteroaryl where the substituents can each independently be hydroxyl, —CO₂H, —CS₂H, —CO₂R, —CS₂R, —COR, —CSR, —CSOH, —CSOR, —COSH, —COSR, —CN, —CONH₂, —CONHR, —CONR₂, —OR, —SR, —O₂CR, —S₂CR, —SOCR and —OSCR;

R₂ can be, for example, hydrogen, —R, —CO₂H, —CS₂H, —CO₂R, —CS₂R, —COR, —CSR, —CSOH, —CSOR, —COSH, —COSR, —CN, —CONH₂, —CONHR, —CONR₂, —OR, —SR, —O₂CR, —S₂CR, —SOCR and —OSCR;

where R can optionally be C₁-C₁₂ unsubstituted linear alkyl, C₃-C₁₂ unsubstituted cyclic alkyl, C₃-C₁₂ unsubstituted branched alkyl, C₆-C₂₀ unsubstituted aryl, C₆-C₂₀ unsubstituted heteroaryl, C₁-C₁₂ substituted linear alkyl, C₃-C₁₂ substituted cyclic alkyl, C₃-C₁₂ substituted branched alkyl, C₆-C₂₀ unsubstituted aryl and C₆-C₂₀ unsubstituted heteroaryl where the substituents can each independently be hydroxyl, —CO₂H, —CS₂H, —CO₂R, —CS₂R, —COR, —CSR, —CSOH, —CSOR, —COSH, —COSR, —CN, —CONH₂, —CONHR, —CONR₂, —OR, —SR, —O₂CR, —S₂CR, —SOCR and —OSCR; such that at least one of R₁-R₄ is not hydrogen.

Examples of suitable ethylenically unsaturated monomers for use in connection with the present teachings include, but are not limited to, N-alkylmaleic anhydrides, N-arylmaleic anhydrides, acrylate esters, methacrylate esters, acrylic acids, methacrylic acids, acrylamides, methacrylamides, styrenes, acrylonitriles, methacrylonitriles, and the like. It will be understood by one of skill in the art that the choice of monomers and/or comonomers is dependent on their reactive ratios, steric and electronic properties, and that selection of particular monomers and/or comonomers can influence the physical properties of the polymer or copolymer formed (e.g.—porosity). A general discussion of polymerization can be found in, for example, Polymer Handbook, 3^{rd} Edition, Brandup, J. and Immergut, E. H., Eds., Wiley, NY (1989).

Examples of suitable ethylenically unsaturated monomers include, but are not limited to, methyl (meth)acrylate, ethyl (meth)acrylate, propyl (meth)acrylate, isopropyl (meth)acrylate, butyl (meth)acrylate, isobutyl (meth)acrylate, sec-butyl (meth)acrylate, tert-butyl (meth)acrylate, amyl (meth)acrylate, hexyl (meth)acrylate, heptyl (meth)acrylate, octyl (meth)acrylate, 2-ethylhexyl (meth)acrylate, isobornyl (meth)acrylate, decyl (meth)acrylate, dodecyl (meth)acrylate, benzyl (meth)acrylate, phenyl (meth)acrylate, methyl α-chloro-acrylate, ethyl α-chloro-acrylate, propyl α-chloro-acrylate, hexyl α-chloro-acrylate, octyl α-chloro-acrylate, decyl α-chloro-acrylate, dodecyl α-chloro-acrylate, dimethyl-acrylamide, diethylacrylamide, dipropylacrylamide, diisopropylacrylamide, (meth)acrylonitrile, 2-(acrylamide)-2-methyl-1-propanesulfonic acid, glycidyl (meth)acrylate, 2-vinyl-4,4-dimethylazalactone and O-(N-succinimido)(meth)acrylate.

In some embodiments, the ethylenically unsaturated monomers can be one or more of methyl (meth)acrylate, ethyl (meth)acrylate, propyl (meth)acrylate, isopropyl (meth)acrylate, butyl (meth)acrylate, isobutyl (meth)acrylate, sec-butyl (meth)acrylate, tert-butyl (meth)acrylate and (meth)acrylonitrile.

Examples of suitable ethylenically unsaturated monomers that introduce reactive sites into the porous copolymer-monolith include, but are not limited to, (meth)acrylic acid, 2-hydroxyethyl (meth)acrylate, 2-hydroxypropyl (meth)acrylate, 3-hydroxypropyl (meth)acrylate, hydroxybutyl (meth)acrylate (all isomers), hydroxysecbutyl (meth)acrylate (all isomers), glycidyl (meth)acrylate, 2-aminoethyl (meth)acrylate, 3-aminopropyl (meth)acrylate, α-chloroacrylic acid, allyl isocyanate, vinyl isocyanate, allyl isothiocyanate, vinyl isothiocyanate, vinyl sulfonic acid, phenyl vinyl sulfonate, methyl vinyl sulfonate and ethyl vinyl sulfonate. Further, porous copolymer-monoliths of the present teachings can comprise additional ethylenically unsaturated monomers such as styrenes and α-methylstyrenes.

Suitable ethylenically unsaturated cross-linkers include those that comprise two or more ethylenically unsaturated moieties. Suitable ethylenically unsaturated cross-linkers include, but are not limited to, diacrylates, dimethacrylates, bisacrylamides, and the like. Examples of ethylenically unsaturated cross-linkers for use in connection with the present teachings include, but are not limited to, ethylene glycol di(meth)acrylate, propylene glycol di(meth)acrylate, 2-ethyl-2-(hydroxymethyl)-1,3-propanediol tri(meth)acrylate, 1,4-butanediol di(meth)acrylate, 1,2-propanediol di(meth)acrylate, 1,3-propanediol di(meth)acrylate, 1,6-hexanediol di(meth)acrylate, pentaerythritol tri(meth)acrylate, dipentaerythritol penta(meth)acrylate, dipentaerythritol hexa(meth)acrylate, tetraethylene glycol di(meth)acrylate, polyethylene glycol di(meth)acrylate, 2,2-bis((meth)acrylamido) acetic acid, N,N′-methylenebis-(meth)acrylamide, N,N′-ethylenebis(meth)acrylamide, N,N′-(1,2-dihydroxyethylene)-bis(meth)acrylamide, N,N′-piperizine di(meth)acrylamide, N,N′-bis(meth)acryloyl methamine, and the like.

In some embodiments, the ethylenically unsaturated monomers comprise acrylic acid and at least one other ethylenically unsaturated monomer. In some embodiments, the ethylenically unsaturated monomers comprise acrylic acid and at least one other acrylate monomer. In some embodiments, the inverse phase photo-copolymerization comprises acrylic acid, at least one other ethylenically unsaturated monomer, and at least one ethylenically unsaturated cross-linker. In some embodiments, the inverse phase photo-copolymerization comprises acrylic acid and at least one ethylenically unsaturated cross-linker. In some embodiments, the inverse phase photo-copolymerization comprises acrylic acid, at least one other acrylate monomer and at least one diacrylate cross-linker. In some embodiments, the inverse phase photo-copolymerization comprises acrylic acid and at least one diacrylate cross-linker. In some embodiments, the inverse phase photo-copolymerization comprises acrylic acid, at least one other acrylate monomer and at least one bisacrylamide cross-linker. In some embodiments, the inverse phase photo-copolymerization comprises acrylic acid and at least one bisacrylamide cross-linker.

In some embodiments, the inverse phase photo-copolymerization comprises acrylic acid and N,N-methylenebisacrylamide. In some embodiments, the inverse phase photo-copolymerization comprises acrylic acid, methyl methacrylate and N,N-methylenebisacrylamide. In some embodiments, the inverse phase photo-copolymerization comprises acrylic acid, butyl acrylate and ethylene glycol diacrylate. In some embodiments, the inverse phase photo-copolymerization comprises acrylic acid, butyl acrylate and poly(ethylene glycol) diacrylate. In some embodiments, the inverse phase photo-copolymerization comprises acrylic acid, methyl acrylate and poly(ethylene glycol) diacrylate.

In some embodiments, prior to polymerization, the ethylenically unsaturated monomers comprise from about 10 to about 98 weight percent (wt %) of acrylic acid. In some embodiments, prior to polymerization, the ethylenically unsaturated monomers comprise from about 10 to about 30 wt % of acrylic acid. In some embodiments, prior to polymerization, the ethylenically unsaturated monomers comprise from about 40 to about 98 wt % of acrylic acid. In some embodiments, prior to polymerization, the ethylenically unsaturated monomers comprise from about 60 to about 90 wt % of acrylic acid. In some embodiments, prior to polymerization, the ethylenically unsaturated monomers comprise from about 10 to about 98 wt % of butyl acrylate. In some embodiments, prior to polymerization, the ethylenically unsaturated monomers comprise from about 30 to about 70 wt % of butyl acrylate. In some embodiments, prior to polymerization, the ethylenically unsaturated monomers comprise from about 30 to about 60 wt % of butyl acrylate. In some embodiments, prior to polymerization, the ethylenically unsaturated monomers comprise from about 50 to about 60 wt % of butyl acrylate. In some embodiments, prior to polymerization, the ethylenically unsaturated monomers comprise from about 10 to about 98 wt % of methyl methacrylate. In some embodiments, prior to polymerization, the ethylenically unsaturated monomers comprise from about 10 to about 50 wt % of methyl methacrylate. In some embodiments, prior to polymerization, the ethylenically unsaturated monomers comprise from about 10 to about 25 wt % of methyl methacrylate. It will be understood that the ranges given above are merely exemplary, and that each range given includes all subranges possible within that range. For example, a range from about 10 to about 20 wt % can include any range using the values 10, 11, 12, 13, 14, 15, 16, 17, 18, 19 and 20 wt % and fractions thereof.

In some embodiments, prior to polymerization, the ethylenically unsaturated cross-linkers comprise from about 1 to about 40 wt % of N,N-methylenebisacrylamide. In some embodiments, prior to polymerization, the ethylenically unsaturated cross-linkers comprise from about 5 to about 30 wt % of N,N-methylenebisacrylamide. In some embodiments, prior to polymerization, the ethylenically unsaturated cross-linkers comprise from about 10 to about 30 wt % of N,N-methylenebisacrylamide. In some embodiments, prior to polymerization, the ethylenically unsaturated cross-linkers comprise from about 10 to about 20 wt % of N,N-methylenebisacrylamide. In some embodiments, prior to polymerization, the ethylenically unsaturated cross-linkers comprise from about 1 to about 40 wt % of ethylene glycol diacrylate. In some embodiments, prior to polymerization, the ethylenically unsaturated cross-linkers comprise from about 10 to about 40 wt % of ethylene glycol diacrylate. In some embodiments, prior to polymerization, the ethylenically unsaturated cross-linkers comprise from about 20 to about 40 wt % of ethylene glycol diacrylate. In some embodiments, prior to polymerization, the ethylenically unsaturated cross-linkers comprise from about 30 to about 40 wt % of ethylene glycol diacrylate. In some embodiments, prior to polymerization, the ethylenically unsaturated cross-linkers comprise from about 1 to about 60 wt % of poly(ethylene glycol) diacrylate. In some embodiments, prior to polymerization, the ethylenically unsaturated cross-linkers comprise from about 20 to about 50 wt % of poly(ethylene glycol) diacrylate. In some embodiments, prior to polymerization, the ethylenically unsaturated cross-linkers comprise from about 30 to about 50 wt % of poly(ethylene glycol) diacrylate.

In some embodiments, the step of copolymerization can be carried out in the presence of at least one initiator. Suitable initiators include, but are not limited to, unimolecular photoinitiators (PI₁), bimolecular photoinitiators (PI₂) and combinations thereof. As used herein, the term “unimolecular photo-initiator” means a single molecule photo-initiator that, upon exposure to visible light, ultraviolet radiation or the like undergoes a unimolecular bond cleavage to form a pair of radicals that can propagate a photo-polymerization as shown in Scheme 1 (unimolecular photoinitiators are often referred to as Type I or homolytic photoinitiators).

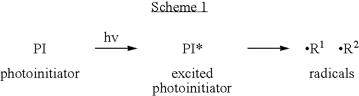

Depending on the nature of the functional group to be fragmented and its location in the molecule, the unimolecular fragmentation can take place at different locations. For example, fragmentation can take place at a bond adjacent to a carbonyl group (sometimes called “α-cleavage”), at a bond one carbon disposed from a carbonyl group (sometimes called “β-cleavage”) or, in the case of particularly weak bonds (like C—S bonds or O—O bonds), elsewhere at a remote position. It will be recognized by those of skill in the art that the most common fragmentation in unimolecular photoinitiator molecules is α-cleavage of the carbon-carbon bond between the carbonyl group and the alkyl residue in an alkyl aryl ketones, which is known as the Norrish Type I reaction.

Examples of unimolecular photoinitiators include, but are not limited to, benzoin ethers, benzoin esters, benzyl ketals, α,α-dialkoxyacetophenones, α-hydroxyalkylphenones, α-aminoalkylphenones α-aminoalkylphosphine, acylphosphine oxides, bisacylphosphine oxides, acylphosphine sulphides, halogenated acetophenone derivatives, and the like. Examples of specific unimolecular photoinitiators include, but are not limited to ethyl benzoin ether, isopropyl benzoin ether, isobutyl benzoin ether, α,α-diethoxyacetophenone, α,α-diethoxy-α-phenylacetophenone, α,α-dimethoxy-α-phenylacetophenone, 4,4′-bis(dimethylamino)benzophenone, ethyl 4-(dimethylamino)-benzoate, 4,4′-dicarboethoxybenzoin ethyl ether, benzoin phenyl ether, α-methylbenzoin ethyl ether, α-methylolbenzoin ethyl ether, α,α,α-trichloroacetophenone, Irgacure 651 (benzildimethyl ketal or 2,2-dimethoxy-1,2-diphenylethanone, Ciba-Geigy), Irgacure 184 (1-hydroxycyclohexylphenyl ketone as the active component, Ciba-Geigy), Darocur 1173 (2-hydroxy-2-methyl-1-phenylpropan-1-one as the active component, Ciba-Geigy), Irgacure 907 (2-methyl-1-[4-(methylthio)phenyl]-2-morpholino propan-1-one, Ciba-Geigy), Irgacure 369 (2-benzyl-2-dimethylamino-1-(4-morpholinophenyl)-butan-1-one as the active component, Ciba-Geigy), Esacure KIP 150 (poly {2-hydroxy-2-methyl-1-[4-(1-methylvinyl)-phenyl]propan-1-one), Fratelli Lamberti), Esacure KIP 100 F (blend of poly {2-hydroxy-2-methyl-1-[4-(1-methylvinyl)phenyl]propan-1-one) and 2-hydroxy-2-methyl-1-phenyl-propan-1-one, Fratelli Lamberti), Esacure KTO 46 (blend of poly{2-hydroxy-2-methyl-1-[4-(1-methylvinyl)phenyl]propan-1-one}, 2,4,6-trimethyl-benzoyl-diphenylphosphine oxide and methylbenzophenone derivatives, Fratelli Lamberti), acylphosphine oxides such as Lucirin TPO (2,4,6-trimethylbenzoyl diphenyl phosphine oxide, BASF), Irgacure 819 (bis(2,4,6-trimethylbenzoyl)-phenylphosphine oxide, Ciba-Geigy), Irgacure 1700 (25:75% blend of bis(2,6-dimethoxybenzoyl)2,4,4-trimethylpentylphosphine oxide and 2-hydroxy-2-methyl-1-phenylpropan-1-one, Ciba-Geigy), and the like.

As used herein, the term “bimolecular photoinitiators” means a pair of molecules that, upon exposure to visible light, ultraviolet radiation or the like undergoes a bimolecular reaction where the excited state of the photoinitiator interacts with a second molecule (a coinitiator) to generate free radicals which can propagate a polymerization. One type of bimolecular photo-initiation involves a hydrogen abstraction process wherein a bimolecular photoinitiator (photosensitizer), upon exposure to, for example, ultraviolet light forms an excited state molecule that can react with a hydrogen donor molecule to produce radicals that can propagate a polymerization, Scheme 2.

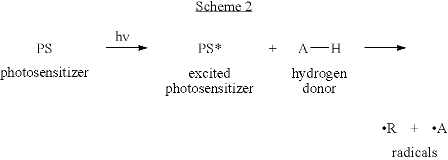

Alternatively, bimolecular photoinitiation can proceed through what is known in the art as an “energy donor” type bimolecular reaction. In this type of process, an excited photosensitizer can transfer energy to another molecule, which can in turn fragment from an excited state into a radical pair, Scheme 3.

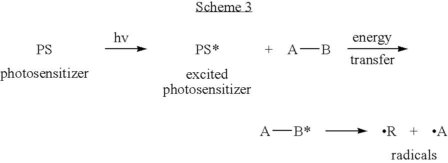

The nature of the molecule to which the excited photosensitizer transfers energy is not particularly limited, and suitable molecules include any molecule that is capable of absorbing the energy donated by the photosensitizer and forming a radical pair in response, including monomers, polymers or added initiators that interact with the photo sensitizer.

Suitable photosensitizers include, but are not limited to aromatic ketones, aromatic aldehydes, thioxanthones or titanocenes. Examples photosensitizers for use in connection with the present teachings include, but are not limited to, benzil, 3,4-benzofluorene, 1-naphthaldehyde, 1-acetylnaphthalene, 2,3-butanedione, 1-benzoyl-naphthalene, 9-acetylphenanthrene, 3-acetylphenanthrene, 2-naphthaldehyde, 2-acetylnaphthalene, 2-benzoylnaphthalene, 2-benzoylnaphthalene, 4-phenylbenzophenone, 4-phenylacetophenone, anthraquinone, thioxanthone, 3,4-methylenedioxyacetophenone, 4-cyanobenzophenone, 4-benzoylpyridine, 2-benzoylpyridine, 4,4′dichlorobenzo-phenone, 4-trifluoromethylbenzophenone, 3-methoxybenzophenone, 4-chlorobenzo-phenone, 3-chlorobenzophenone, 3-benzoylpyridine, 4-methoxybenzophenone, 3,4-dimethylbenzophenone, 4-methylbenzophenone, benzophenone, 2-methylbenzophenone, 4,4′dimethylbenzophenone, 2,5-dimethyl-benzophenone, 2,4-dimethylbenzophenone, 4-cyanoacetophenone, 4-fluoro-benzophenone, o-benzoylbenzophenone, 4,4′-dimethoxy-benzophenone, 4-acetylpyridine, 3,4,5-trimethylacetophenone, 4-methoxybenzaldehyde, 4-methylbenzaldehyde, 3,5-dimethylacetophenone, 4-bromoacetophenone, 4-methoxy-acetophenone, 3,4-dimethylacetophenone, benzaldehyde, triphenylmethylacetophenone, anthrone, 4-chloroacetophenone, 4-trifluoromethylacetophenone, 2-chloroanthraquinone, ethyl phenylglyoxylate, o-benzoylbenzoic acid, ethyl benzoylbenzoate, dibenzosuberone, o-benzoylbenzophenone, and the like.

It is known to those of skill in the art that photopolymerizations involving, for example, monomers containing acrylic groups are often inhibited by oxygen and thus such polymerizations should not be performed open to the air. However, it is also known in the art that by combining unimolecular and bimolecular photoinitiators that oxygen inhibition can be greatly reduced. Not to be bound by any particular theory or hypothesis, it is believed by those of skill in the art that PI₁/PI₂ combination photoinitiation may take place through a process similar to that shown in Scheme 4.

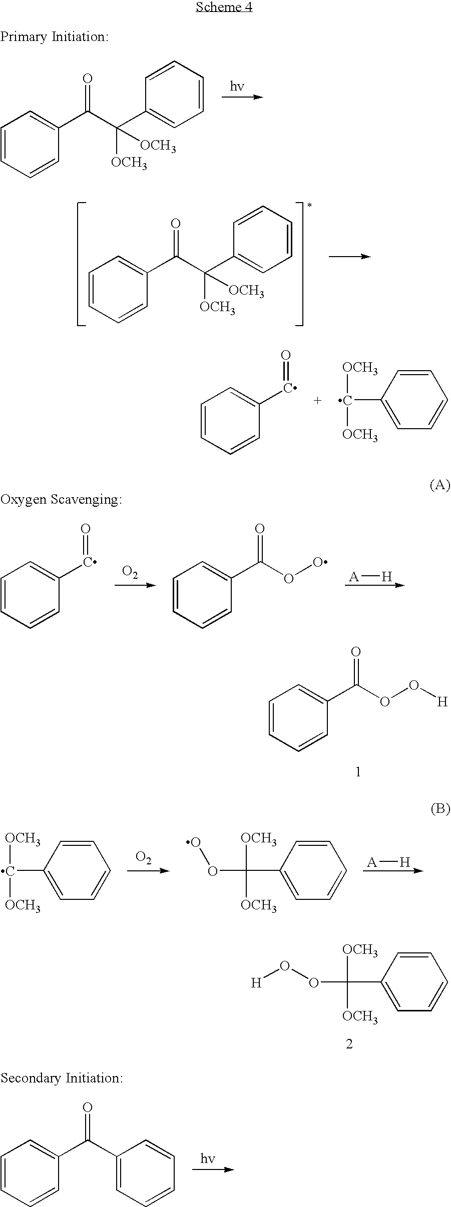

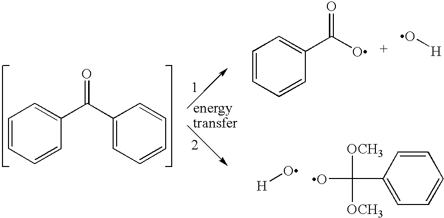

Using α,α-dimethoxy-α-phenylacetophenone as an example of a photoinitiator, as shown in Scheme 4, α,α-dimethoxy-α-phenylacetophenone can become excited by light and can fragment into a pair of carbon-centered free radicals. These carbon-centered free radicals can initiate free radical polymerization and also act as oxygen scavengers to form a pair of O₂ radicals. Alone, these O₂ radicals could not further initiate free radical polymerization, however, in the presence of a proton donor, these O₂ radicals can form hydrogen peroxides (1) and (2). Polymerization can then occur via interaction of excited benzophenone molecules (i.e.—a photosensitizer) with hydrogen peroxides (1) and (2) to form oxygen-centered free radicals that are capable of initiating free radical polymerization. It is known to those of skill in the art that the combination PI₁ and PI₂ polymerization initiators provides an oxygen scavenging process that enables free radical polymerization to be carried out open to the air. It will be understood by those of skill in the art that a variety of PI₁/PI₂ photoinitiators are suitable for use in connection with the present teachings. Further guidance can be found in, for example, Gruber, G. W., U.S. Pat. No. 4,017,652; Gruber, G. W., U.S. Pat. No. 4,024,296; Barzynski, et al., U.S. Pat. No. 4,113,593; Ng, et al., Macromolecules, v. 11, p. 937 (1978); Wismontski-Knittel, et al., J. Polymer Sci: Polymer Chem. Ed., v. 21, p. 3209 (1983).

In some embodiments, the present teachings can provide a composite substrate comprising a porous copolymer-monolith covalently attached to a surface of a substrate, wherein the porous copolymer-monolith has been formed by an inverse phase photo-copolymerization process comprising photo-copolymerizing at least one ethylenically unsaturated monomer with polymerizable surface functionalities that are covalently attached to a surface of a derivitized substrate such that, after photo-copolymerization, the porous copolymer-monolith is covalently attached to the surface of the substrate, and wherein the photo-copolymerizing is carried out in the presence of at least one porogenic solvent. In some embodiments, the inverse phase photo-polymerization process can be carried out using a unimolecular photoinitiator, using a bimolecular photoinitiator or using a unimolecular bimolecular combination photoinitiator. In some embodiments, the inverse phase photo-polymerization process can be carried out using a unimolecular photoinitiator. In some embodiments, the inverse phase photo-polymerization process can be carried out using a bimolecular photoinitiator. In some embodiments, the inverse phase photo-polymerization process can be carried out using a unimolecular/bimolecular combination photoinitiator.

In some embodiments, the present teachings provide for methods of fabricating a composite substrate comprising:

i) contacting a derivitized substrate having polymerizable surface functionalities covalently attached thereto with a solution comprising at least one porogenic solvent, at least one photopolymerization initiator, and at least one ethylenically unsaturated monomer; and

ii) copolymerizing the polymerizable surface functionalities with the at least one ethylenically unsaturated monomer to form a porous-copolymer monolith covalently attached to a substrate. In some embodiments, the solution further comprises at least one ethylenically unsaturated cross-linker.

In some embodiments, the photo-polymerization initiator can be a unimolecular photoinitiator, a bimolecular photoinitiator or a unimolecular/bimolecular combination photoinitiator. In some embodiments, the photo-polymerization initiator can be a unimolecular photoinitiator. In some embodiments, the photo-polymerization initiator can be a bimolecular photoinitiator. In some embodiments, the photo-polymerization initiator can be a unimolecular/bimolecular combination photoinitiator.

As used herein, the terms “porogenic solvent” and “porogen” are used interchangeably and refer to any solvent that is capable of inducing porosity in photopolymerized polymers. Porogens are generally categorized by their dielectric constants, where it is generally understood that a porogen having a high dielectric constant (i.e.—a fairly polar solvent) can lead to more macroporous polymers having a larger mean pore diameter. On the contrary, solvents of having a low dielectric constant (i.e.—a relatively non-polar solvent) can lead to polymers having a lower macroporosity. For example, acetonitrile (dielectric constant, e=36) can be considered a polar solvent that would lead to more macroporous polymers, and chloroform (e=5) can be considered a non-polar solvent that would lead to less macroporous polymers.

Suitable porogenic solvents for use in connection with the present teachings include, but are not limited to, any organic solvent or mixture of solvents from which a porous polymer monolith is formed as the porogenic solvent or mixture of porogenic solvents phase-separates to form the discrete phase (inverse phase polymerization) during a polymerization. Examples of solvents include ethers, such as ethyl ether, isopropyl ether, butyl ether, and the like, hydrocarbons, such as fully saturated hydrocarbon solvents having from 1-20 carbon atoms, unsaturated hydrocarbons having from 4-19 carbon atoms or cyclic hydrocarbons having from 4-20 carbon atoms, and ketones, such as dialkyl ketones, aryl alkyl ketones, diaryl ketones, and the like. As used herein, the term “saturated hydrocarbon” includes branched and unbranched hydrocarbons. Examples of saturated hydrocarbons include, but are not limited to n-pentane, neopentane, n-hexane, 2-ethylbutane, 2-methylpentane, 3-methylpentane, heptane, 2-methylhexane, 3-methylhexane, 3-ethylpentane, 2,3-dimethyl pentane, octane, isooctane, nonane, decane, undecane, dodecane, tridecane, tetradecane, pentadecane, hexadecane, heptadecane, octadecane, and the like. As used herein, the term “unsaturated hydrocarbon” includes branched and unbranched hydrocarbons having at least one site of unsaturation, that is at least one carbon-carbon bond between carbon atoms in an sp² orbital hybridization or at least one carbon-carbon bond between carbon atoms in an sp orbital hybridization. Examples of unsaturated hydrocarbons include 2-methyl-2-butene, etc. As used herein, the term “cyclic hydrocarbon” means any saturated or unsaturated hydrocarbon having at least one carbocyclic ring. “Cyclic hydrocarbon”, as used herein, can also encompass carbocyclic compounds wherein one or more of the carbon atoms is replaced by a heteroatom selected from S, N and O. Examples of cyclic hydrocarbons include, but are not limited to, cyclohexane, cyclooctane, cyclohexene, cyclooctene, benzene, toluene, pyridine, thiophene, furan, and the like.

It will be understood that as used herein, “hydrocarbon solvents” also include those commonly known in the art that are derived from petroleum fractions. It will be further understood that hydrocarbon solvents can be mixtures of molecules that differ in structure and molecular weight, and thus are often characterized on a performance basis (i.e.—boiling range, flash point, etc.). Some hydrocarbon solvents, such as white spirit, can be relatively easily obtained from selected crude oil by simple distillation (and desulphurization). Other hydrocarbon solvent types require more processing steps, such as hydrogenation and fractionation. For example, commonly known isoparaffins are typically chemically synthesized. In general, hydrocarbon solvents can include, but are not limited to, isoparaffins, cycloparaffins, aliphatics (from fast evaporating to high flash point mineral spirits), aromatics and blends.

In some embodiments, the at least one solvent can be pentadecane, 2-butanone, dioxane, heptane, ethyl ether, etc. or any combination thereof. In some embodiments, the at least one solvent can be pentadecane. In some embodiments, the at least one solvent can be 2-butanone.

In some embodiments, the step of copolymerizing can be carried out in conjunction with exposure of the solution to light. In some embodiments, the step of copolymerizing can be carried out in conjunction with exposure to UV light.

It will be understood by those of skill in the art that a number of variables can have an effect on the porous properties of porous polymers that are prepared using photopolymerization including, irradiation time, lamp power, percentage of cross-linker, relative percentages of monomers, monomer identity, concentration of initiator and composition and percentage of porogen. See, for example, Yu, C., et al., J. Polym. Sci., Polym. Chem., 40(6), 755-769 (2002), Svec, F., et al., Ind. Eng. Chem. Res, v.38, 34-48 (1999), Guyot, A., et al., Prog. Polym Sci, v.8, 277 (1982) and Seidl, J., et al., Adv. Polym. Sci., v.5, 11 (1967) each of which is incorporated herein by reference in its entirety. It will be further understood by one of skill in the art that measuring and evaluating polymer materials for average pore size, porosity and/or surface area is well known in the art. Porosity can be evaluated visually by scanning electron microscopy to obtain images of porous polymers using, for example, a Hitachi s2400 electron microscope. Porosity can also be measured by methods such as mercury porosimetry, gas adsorption ellipsometric porosimetry, and x-ray porosimetry using a variety of commercially available systems. In some embodiments, the present teachings provide for composite substrates comprising a porous copolymer monolith covalently attached thereto, wherein the porous copolymer monolith has been formed by inverse phase polymerization and can have an average pore size of from about 0.01 μm to about 100 μm. In some embodiments, the average pore size can be from about 0.01 μm to about 20 μm. In some embodiments, the average pore size can be from about 0.01 μm to about 10 μm. In some embodiments, the average pore size can be from about 0.01 μm to about 1.0 μm. In some embodiments, the average pore size can be from about 0.01 μm to about 0.5 μm.

In some embodiments, the porous copolymer monolith can have a porosity of from about 10% to about 95%. In some embodiments, the porous copolymer monolith can have a porosity of from about 10% to about 65%. In some embodiments, the porous copolymer monolith can have a porosity of from about 10% to about 35%.

In some embodiments, composite substrates of the present teachings further comprise at least one pigment. The addition of pigments can be advantageous for certain applications. For example, fluorescence background (i.e.—autofluorescence can be detrimental to the sensitivity of fluorescence detection systems in, for example, microarray applications. In such systems, high and/or variable background fluorescence can have adverse effects on the efficiency of hybridization signal across a microarray, thus reducing the dynamic range achievable by the microarray and/or increasing the variation of signal ratios. As a result of high and/or variable background, the detection of genes expressed at low levels in a sample can become problematic.

Similarly, it can also be advantageous to include pigments in microarray substrates in systems where chemiluminescence is employed as a detection method. Specifically, reflectance of signals in chemiluminescence systems can reduce sensitivity and dynamic range while increasing variations in signal ratios on the microarray. As a result, reflectance can make resolution of genes having only slight differences in signal intensity problematic. In addition, reflectance generated from features having intense signals (i.e.—from features having high gene expression levels) can obscure neighboring features and can result in increased overall background through distribution of intense signals across the entire microarray. As such, the addition of pigments can be advantageous. Suitable pigments for use in connection with the present teachings can include any of a variety of carbon blacks that are known in the art. It will be understood by those of skill in the art that there are a variety of carbon black fillers that can be selected for use in connection with the present teachings and there are various techniques for dispersing carbon black formulations into polymers and polymerization formulations in order to obtain the desired tinting affect.

In some embodiments, the present teachings provide for composite substrates of the type described above having at least one biomolecule covalently attached thereto. In some embodiments, the biomolecule can be a polynucleotide, a protein, a peptide, a peptide nucleic acid (PNA) and a PNA/DNA chimera. In some embodiments, the present teachings provide for a composite substrate of the type described above having a plurality of polynucleotides covalently attached thereto in a spatially addressable manner. In some embodiments, the present teachings provide for a microarray comprising a composite substrate of the present teachings having a plurality of polynucleotides covalently attached thereto in spatially addressable features.

It will be understood by those of skill in the art that biomolecule conjugation to composite substrates of the present teachings can be carried out using a variety of methods known in the art. For example, as described above, polynucleotides can be covalently attached to a substrate by in situ synthesis of polynucleotides, see for example Fodor, et al., U.S. Pat. No. 5,424,186; Pirrung, et al., U.S. Pat. No. 5,143,854 and Bass, et al. U.S. Pat. No. 6,440,669. Alternatively, as described above, pre-synthesized polynucleotides can be covalently attached to a substrate surface using known chemistries, see for example, Okamoto, T., et al., U.S. Pat. No. 6,476,215; Bruhn, et al., U.S. Pat. No. 6,458,853 and Southern, E., U.S. Pat. No. 5,700,637.

As used herein, the terms “oligonucleotide”, “polynucleotide” and “nucleic acid” are used interchangeably to refer to single- or double-stranded polymers of DNA, RNA or both, including polymers containing modified or non-naturally occurring nucleotides. In addition, the terms oligonucleotide, polynucleotide and nucleic acid refer to any other type of polymer comprising a backbone and a plurality of nucleobases that can form a duplex with a complimentary polynucleotide strand by nucleobase-specific base-pairing, including, but not limited to, PNA/DNA chimeras, bicyclo DNA oligomers (Bolli, et al., Nucleic Acids Res. 24:4660-4667 (1996)) and related structures.

In some embodiments, polynucleotides can comprise a backbone of naturally occurring sugar or glycosidic moieties, for example, β-D-ribofuranose. In addition, in some embodiments, modified nucleotides of the present teachings can comprise a backbone that includes one or more “sugar analogs”. As used herein, the term “sugar analog” refers to analogs of the sugar ribose. Exemplary ribose sugar analogs include, but are not limited to, substituted or unsubstituted furanoses having more or fewer than 5 ring atoms, e.g., erythroses and hexoses and substituted or unsubstituted 3-6 carbon acyclic sugars. Typical substituted furanoses and acyclic sugars are those in which one or more of the carbon atoms are substituted with one or more of the same or different —R, —OR, —NRR or halogen groups, where each R independently comprises —H, (C₁-C₆) alkyl or (C₃-C₁₄) aryl.

Examples of unsubstituted and substituted furanoses having 5 ring atoms include but are not limited to 2′-deoxyribose, 2′-(C₁ C₆)-alkylribose, 2′-(C₁-C₆)— alkoxyribose, 2′-(C₅-C₁₄)-aryloxyribose, 2′,3′-dideoxyribose, 2′,3′-dideoxy-ribose, 2′-deoxy-3′-haloribose, 2′-deoxy-3′-fluororibose, 2′-deoxy-3′-chlororibose, 2′-deoxy-3′-aminoribose, 2′-deoxy-3′-(C₁-C₆)-alkylribose, 2′-deoxy-3′-(C₁-C₆)-alkoxy-ribose, 2′-deoxy-3′-(C₅-C₁₄)-aryloxyribose, 3′-(C₁-C₆)-alkylribose-5′-triphosphate, 2′-deoxy-3′-(C₁-C₆)-alkylribose-5′-triphosphate, 2′-deoxy-3′-(C₁-C₆)-alkoxyribose-5′-triphosphate, 2′-deoxy-3′-(C₅-C₁₄)-aryloxyribose-5′-triphosphate, 2′-deoxy-3′-haloribose-5′-tri-phosphate, 2′-deoxy-3′-aminoribose-5′-triphosphate, 2′,3′-dideoxy-ribose-5′-triphosphate or 2′,3′-didehydroribose-5′-triphosphate. Further sugar analogs include but are not limited to, for example “locked nucleic acids” (LNAs), i.e., those that contain, for example, a methylene bridge between C-4′ and an oxygen atom at C-2′, such as 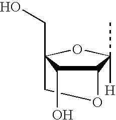
described in Wengel, et al. WO 99/14226, incorporated herein by reference, and Wengel J., Acc. Chem. Res., 32:301-310 (1998).

In some embodiments, polynucleotides of the present teachings include those in which the phosphate backbone comprises one or more “phosphate analogs”. The term “phosphate analog” refers to analogs of phosphate wherein the phosphorous atom is in the +5 oxidation state and one or more of the oxygen atoms are replaced with a non-oxygen moiety. Exemplary analogs include, but are not limited to, phosphorothioate, phosphorodithioate, phosphoroselenoate, phosphorodiselenoate, phosphoroanilothioate, phosphoranilidate, phosphoramidate, boronophosphates, and associated counterions, including but not limited to H⁺, NH4⁺, Na⁺, Mg⁺⁺ if such counterions are present. Further polynucleotide analogs include those containing phosphate analogs such as phosphorothioate linkages, methylphosphonates and/or phosphoroamidates (see, Chen et al., Nucl. Acids Res., 23:2662-2668 (1995)). Combinations of polynucleotide linkages are also within the scope of the present teachings.

As used herein, the term “polynucleotides” also includes DNA/PNA chimeras. Peptide nucleic acids (PNAs, also known as polyamide nucleic acids), see, for example, Nielsen et al., Science 254:1497-1500 (1991), contain heterocyclic nucleobase units that are linked by a polyamide backbone instead of the sugar-phosphate backbone characteristic of DNA and RNA. PNAs are capable of hybridization to complementary DNA and RNA target sequences. Synthesis of PNA oligomers and reactive monomers used in the synthesis of PNA oligomers are described in, for example, U.S. Pat. Nos. 5,539,082; 5,714,331; 5,773,571; 5,736,336 and 5,766,855. Alternate approaches to PNA and DNA/PNA chimera synthesis and monomers for PNA synthesis have been summarized in, for example, Uhlmann, et al., Angew. Chem. Int. Ed. 37:2796-2823 (1998).

In some embodiments, polynucleotides for use in connection with the present teachings can range in size from a few nucleotide monomers in length, e.g. from 5 to 100, to hundreds of nucleotide monomers in length. For example, polynucleotides can contain from 5 to 80 nucleotides, 20 to 80 nucleotides, or 30 to 80 nucleotides. When, in some embodiments, polynucleotides contain, for example, from 30 to 80 nucleotides, such a range includes all possible ranges of integers between 30 an 80, for example 30, 35, 40, 45, 50, 55, 60, 65, 70, 75 and 80 nucleotides in length. Whenever a polynucleotide is represented by a sequence of letters, such as “ATGCCTG,” it will be understood that the nucleotides are in 5′ to 3′ order from left to right and that “A” denotes deoxyadenosine, “C” denotes deoxycytidine, “G” denotes deoxygaunosine, and T denotes thymidine, unless otherwise indicated. Additionally, whenever a polynucleotide is represented by a sequence of letters that includes an “X”, it will be understood that the “X” denotes a variable nucleotide monomer, where “X” is a nucleotide monomer other than “A”, “C”, “G” or “T”.

It will be understood that the following examples are meant to be merely illustrative and are not meant to be limiting of the present teachings in any way. Although the above description will be adequate to teach one of skill in the art how to practice the present teachings, the following examples are provided as further guidance to those of skill in the art.

### EXAMPLES

Materials and Methods:

Unless otherwise indicated, all chemicals and solvents were obtained from Aldrich Chemical (Milwaukee, Wis.) and were used as received from the distributor. Acrylic acid (99%) was redistilled prior to use. Methyl-3-(dimethylamino)benzoate (99+%) was obtained from TCI America (Portland, Oreg.). (Hexadecafluoro-1,1,2,2-tetrahydrodecyl)triethoxysilane, 3-(dimethoxymethylsilyl)propyl methacrylate and 3-(dimethoxymethylsilyl)propyl acrylate were obtained from Gelest (Morrisville, Pa.). Polymerization was carried out by exposure of the polymerization mixture to UV light using a Spectroline® BIB-150P Series 150-W long wave UV lamp (Spectronics Corporation, Westbury, N.Y.). Glass microscope slide were obtained from VWR International (Bristol, Conn.).

### Example 1

Glass Slide Fluorosilylation:

A glass microscope slide was cleaned in an ultrasonic bath with 1% SDS aqueous solution for 20 minutes, followed by sonication in 4% hydrofluoric acid for 10 minutes. The slides were then dried in an oven at 110° C. for 60 minutes and cooled to room temperature in a ventilation hood prior to use.

The glass slide was then immersed in a 2% solution of (hexadecafluoro-1,1,2,2-tetrahydrodecyl)triethoxysilane in a 95% ethanol solution (pH adjusted to 4.5-5.5 (measured via pH Indicator Sticks, J. T. Baker (Paris, Ky.)) prior to immersion by adding 1 Molar (1M) acetic acid (AcOH) dropwise with constant stirring for 10 minutes)) at room temperature for 5 minutes with occasional agitation. The glass slide was removed from the silylation solution, rinsed twice by dipping briefly in fresh 95% ethanol (EtOH) and cured by heating in a 110° C. oven for 30 minutes to provide a fluorosilylated glass slide for use as a passivated cover-slip.

Glass Slide Methacryloxysilylation:

A glass microscope slide was sonicated in a 2% SDS aqueous solution for 30 minutes. The slide was rinsed thoroughly with Milli-Q water after the sonication. The rinsed slide was then sonicated in a 6 Normal (6N) hydrochloric acid (HCl) solution for 30 minutes, and again rinsed thoroughly with Milli-Q water after sonication. The rinsed slide was finally sonicated in a 10% sodium hydroxide (NaOH) aqueous solution, and again rinsed thoroughly after sonication. The treated slide was then dried in a 100° C. oven for 60 minutes and allowed to cool to room temperature prior to use.

The treated glass slide was immersed in a silylation solution of 120 g methanol (MeOH) 8.0 g of 0.5 mM AcOH and 3.11 g of 3-(dimethoxymethylsilyl)propyl methacrylate for 10 minutes at room temperature. The slides were removed, rinsed with acetone, then rinsed with water and finally cured in an oven at 110° C. for 10 minutes. The silylated slides were allowed to cool to room temperature prior to use.

Photo-Copolymerization

A gasket was fabricated by forming a trough 15.0 mm×15.0 mm×30.0 μm on a methacryloxysilylated glass slide using 3M Scotch tape (3M, St. Paul, Minn.). To the trough was added 60 μL of a pre-formed solution containing 0.3 mL of methyl ethyl ketone and 0.3 mL of a solution containing 81.25 wt % acrylic acid, 13.38 wt % N,N-methylenebisacrylamide, 2.6 wt % benzophenone, and 2.78 wt % methyl 3-(dimethylamino)benzoate. The trough was covered with the fluorosilylated glass slide from above as a cover slip. The resulting microscope slide assembly was placed under a 150-Watt long wavelength UV lamp at a distance of 6 inches. The lamp was turned on and the microscope slide assembly was illuminated for 5 minutes. The light was then turned off and the microscope slide assembly was allowed to stand for 10 minutes at room temperature.

The cover slip was removed to reveal a milky white porous monolith copolymer that was covalently bound to the surface of the methacryloxysilylated glass slide. To test the stability of the monolith, the slide was soaked in ethyl acetate at 35° C. for 4 days. No delamination was observed.

### Example 2

Glass Slide Fluorosilylation

A glass microscope slide was cleaned in an ultrasonic bath with 1% SDS aqueous solution for 30 minutes, rinsed with deionized water, then immersed in 6M HCl for 60 minutes and rinsed with deionized water. The glass slide was then immersed in 10% aqueous NaOH solution for 2 days at room temperature and rinsed with deionized water. The slides were then dried in an oven at 110° C. for 3 hours and cooled to room temperature in a ventilation hood prior to use.

The glass slide was then immersed in a stirred solution of 100 mL of 50% ethanol and 1.9418 g of (hexadecafluoro-1,1,2,2-tetrahydrodecyl)triethoxysilane (pH adjusted to about 4.0 via pH Indicator Sticks prior to immersion by adding 0.8112 g of glacial acetic acid (AcOH) with constant stirring for 10 minutes) at room temperature for 10 minutes with occasional agitation. The glass slide was removed from the silylation solution, rinsed twice with 95% ethanol (EtOH) and cured by heating in an oven at 110° C. for 30 minutes to provide a fluorosilylated glass slide for use as a passivated cover-slip.

Glass Slide Methacryloxysilylation:

A glass microscope slide was sonicated in a 2% SDS aqueous solution for 30 minutes. The slide was rinsed thoroughly with Milli-Q water after the sonication. The rinsed slide was then sonicated in a 6 Normal (6N) hydrochloric acid (HCl) solution for 30 minutes, and again rinsed thoroughly with Milli-Q water after sonication. The rinsed slide was finally sonicated in a 10% sodium hydroxide (NaOH) aqueous solution, and again rinsed thoroughly after sonication. The treated slide was then dried in a 100° C. oven for 60 minutes and allowed to cool to room temperature prior to use.

The treated glass slide was immersed in a silylation solution of 120 g methanol (MeOH) 8.0 g of 0.5 mM AcOH and 3.11 g of 3-(dimethoxymethylsilyl)propyl methacrylate for 10 minutes at room temperature. The slides were removed, rinsed with acetone, then rinsed with water and finally cured in an 110° C. oven for 10 minutes. The silylated slides were allowed to cool to room temperature prior to use.

Photo-Copolymerization

A gasket was fabricated as above. To the trough was added 20 μL of a pre-formed solution containing 10 μL of pentadecane and 10 μL of a solution containing 12.25 wt % acrylic acid, 50.61 wt % butyl acrylate, 25.19 wt % ethylene glycol diacrylate, 1.86 wt % benzophenone, and 1.80 wt % ethyl 4-(dimethylamino)benzoate. The trough was covered with the fluorosilylated glass slide from above as a cover slip. The microscope slide assembly was placed under a 150-Watt long wavelength UV lamp at a distance of 6 inches. The lamp was turned on and the microscope slide assembly was illuminated for 5 minutes. The light was then turned off and the microscope slide assembly was allowed to stand for 10 minutes at room temperature.

The cover slip was removed to reveal a milky white porous monolith copolymer that was covalently bound to the surface of the methacryloxysilylated glass slide and withstood delamination tests.

### Example 3

Glass Slide Acryloxysilylation:

A glass microscope slide was sonicated in a 1% SDS aqueous solution for 15 minutes. The slide was rinsed thoroughly with Milli-Q water after the sonication and dried at 110° C. The rinsed slide was then sonicated in Pirahna solution (a mixture of 70% v/v concentrated sulfuric acid (98 wt %) and 30% v/v hydrogen peroxide solution (30 wt %)) for 60 minutes, and again rinsed thoroughly with Milli-Q water after sonication. The treated slide was then dried in a 110° C. oven for 10 minutes and allowed to cool to room temperature prior to use.

The treated glass slide was immersed in a silylation solution of 120 mL MeOH, 40 μL of 1.0M AcOH and 3.00 mL of 3-(dimethoxymethylsilyl)propyl acrylate for 10 minutes at room temperature. The slides were removed, rinsed with acetone, and allowed to stand at room temperature overnight.

Photo-Copolymerization

A gasket was fabricated on a polytetrafluoroethylene (PTFE) block using 3M Scotch tape forming a trough 15.0 mm×15.0 mm×30.0 μm. To the trough was added 20 μL of a pre-formed solution containing 10 μL of pentadecane and 10 μL of a solution containing 12.37 wt % acrylic acid, 50.81 wt % of butyl acrylate, 33.14 wt % ethylene glycol diacrylate, 1.86 wt % benzophenone, and 1.82 wt % ethyl 4-(dimethylamino)benzoate. The trough was covered with the acryloxysilylated glass slide from above. The PTFE block/slide assembly was placed under a 150-Watt long wavelength UV lamp at a distance of 6 inches away from the light source. The lamp was turned on and the microscope slide assembly was illuminated for 5 minutes. The light was then turned off and the microscope slide assembly was allowed to stand for 10 minutes at room temperature.

The glass microscope slide was removed from the PTFE block to reveal an opaque white porous monolith copolymer that was covalently bound to the surface of the acryloxysilylated glass slide and withstood delamination tests.

### Example 4

Glass Slide Acryloxysilylation:

A glass slide was acryloxysilylated as in Example 3.

Photo-Copolymerization

A gasket was fabricated on a polytetrafluoroethylene (PTFE) block as in Example 3. To the trough was added 20 μL of a pre-formed solution containing 10 μL of pentadecane and 10 μL of a solution containing 13.09 wt % acrylic acid, 56.10 wt % of butyl acrylate, 26.92 wt % ethylene glycol diacrylate, 2.01 wt % benzophenone, and 1.89 wt % ethyl 4-(dimethylamino)benzoate. The trough was covered with the acryloxysilylated glass slide from above. The PTFE block/slide assembly was placed under a 150-Watt long wavelength UV lamp at a distance of 6 inches away from the light source. The lamp was turned on and the microscope slide assembly was illuminated for 5 minutes. The light was then turned off and the microscope slide assembly was allowed to stand for 10 minutes at room temperature.

The glass microscope slide was removed from the PTFE block to reveal an opaque white porous monolith copolymer that was covalently bound to the surface of the acryloxysilylated glass slide and withstood delamination tests.

5× serial dilutions of anti-DIG alkaline phosphatase were prepared in PBS-Tween. 5 μL aliquots were spotted onto the monolith surface in triplicate. Spots were allowed to dry over 3 days at room temperature to bind alkaline phosphatase by physisorption. The surface was wetted with Tris-HCl, and then a chemiluminescence reaction was initiated by the addition of TFE-CDPStar (Applied Biosystems, Foster City, Calif.), and emitted chemiluminescence light was measured by CCD imaging. Luminescence Enhancer Solution (Applied Biosystems, Foster City, Calif.) was added to the composite substrate and the substrate was imaged again using CCD imaging. The composite substrate gave a positive chemiluminescence image.

### Example 5

Glass Slide Acryloxysilylation:

A glass slide was acryloxysilylated as in Example 3.

Photo-Copolymerization

A gasket was fabricated on a polytetrafluoroethylene (PTFE) block as in Example 3. To the trough was added 20 μL of a pre-formed solution, which had been vortexed for 5 minutes prior to use, containing 3.0 mL of pentadecane, 3.0 mg of Raven 5000 Ultra (carbon black powder obtained from Columbian Chemicals, Akron, Ohio), 40.0 mg of Span-80 and 7.0 mL of a solution containing 18.28 wt % acrylic acid, 38.86 wt % of butyl acrylate, 39.49 wt % poly(ethylene glycol) diacrylate, 1.71 wt % benzophenone, and 1.67 wt % ethyl 4-(dimethylamino)benzoate. The trough was covered with the acryloxysilylated glass slide from above. The PTFE block/slide assembly was placed under a 150-Watt long wavelength UV lamp at a distance of 6 inches away from the light source. The lamp was turned on and the microscope slide assembly was illuminated for 5 minutes. The light was then turned off and the microscope slide assembly was allowed to stand for 10 minutes at room temperature.

The glass microscope slide was removed from the PTFE block to reveal an opaque grey porous monolith copolymer.

1. A composite substrate comprising a porous copolymer-monolith covalently attached to a surface of a substrate, wherein the porous copolymer-monolith has been formed by an inverse phase photo-copolymerization process comprising photo-copolymerizing at least one ethylenically unsaturated monomer with polymerizable surface functionalities that are covalently attached to a surface of a derivitized substrate such that, after photo-copolymerization, the porous copolymer-monolith is covalently attached to the surface of the substrate, and wherein the photo-copolymerizing is carried out in the presence of at least one porogenic solvent.

2. The composite substrate of claim 1, wherein the substrate is selected from a polymer substrate, plastic and glass.

3. The composite substrate of claim 2, wherein the substrate is glass.

4. The composite substrate of claim 1, wherein the derivitized substrate comprises an organo-silane selected from 3-(trimethoxysilyl)propyl acrylate, 3-(trimethoxy-silyl)propyl methacrylate, 3-(triethoxysilyl)propyl acrylate, 3-(triethoxysilyl)propyl methacrylate, 3-(dimethoxymethylsilyl)propyl acrylate, 3-(dimethoxymethylsilyl)propyl methacrylate, 3-(diethoxymethylsilyl)propyl acrylate, 3-(diethoxymethylsilyl)propyl methacrylate, 3-(methoxydimethylsilyl)propyl acrylate, 3-(methoxydimethylsilyl)propyl methacrylate, 3-(ethoxydimethylsilyl)propyl acrylate and 3-(ethoxydimethylsilyl)propyl methacrylate, and combinations thereof.

5. The composite substrate of claim 1, wherein the organo-silane is 3-(dimethoxymethylsilyl)propyl methacrylate.

6. The composite substrate of claim 1, wherein the organo-silane is 3-(dimethoxymethylsilyl)propyl acrylate.

7. The composite substrate of claim 1, wherein the at least one ethylenically unsaturated monomer is selected from acrylic acid, butyl acrylate, methyl methacrylate, methyl acrylate and combinations thereof.

8. The composite substrate of claim 1, wherein the at least one ethylenically unsaturated monomer comprises acrylic acid and at least one other ethylenically unsaturated monomer.

9. The composite substrate of claim 1, wherein the inverse phase photo-copolymerization further comprises at least one ethylenically unsaturated cross-linker that contains two or more ethylenically unsaturated moieties.

10. The composite substrate of claim 9, wherein the at least one ethylenically unsaturated monomer is acrylic acid.

11. The composite substrate of claim 9, wherein the at least one ethylenically unsaturated monomer is selected from acrylic acid, methyl methacrylate, methyl acrylate, butyl acrylate, and combinations thereof, and the ethylenically unsaturated cross-linker can be selected from ethylene glycol diacrylate, poly(ethylene glycol) diacrylate, N,N-methylenebisacrylamide and combinations thereof.

12. The composite substrate of claim 11, wherein the at least one ethylenically unsaturated monomers are acrylic acid and the ethylenically unsaturated cross-linker is N,N-methylenebisacrylamide.

13. The composite substrate of claim 11, wherein the at least one ethylenically unsaturated monomer are acrylic acid and methyl methacrylate and the ethylenically unsaturated cross-linker is N,N-methylenebisacrylamide.

14. The composite substrate of claim 11, wherein the at least one ethylenically unsaturated monomer are acrylic acid and butyl acrylate and the ethylenically unsaturated cross-linker is ethylene glycol diacrylate.

15. The composite substrate of claim 11, wherein the at least one ethylenically unsaturated monomer are acrylic acid and butyl acrylate and the ethylenically unsaturated cross-linker is poly(ethylene glycol) diacrylate.

16. The composite substrate of claim 1, wherein the at least one ethylenically unsaturated monomer are acrylic acid and methyl acrylate and the ethylenically unsaturated cross-linker is poly(ethylene glycol) diacrylate.

17. The composite substrate of claim 1, wherein prior to polymerization, the at least one ethylenically unsaturated monomer comprises from about 10 to about 30 wt % of acrylic acid.

18. The composite substrate of claim 1, wherein prior to polymerization, the at least one ethylenically unsaturated monomer comprises from about 40 to about 98 wt % of acrylic acid.

19. The composite substrate of claim 1, wherein prior to polymerization, the at least one ethylenically unsaturated monomer comprises from about 60 to about 90 wt % of acrylic acid.

20. The composite substrate of claim 1, wherein prior to polymerization, the at least one ethylenically unsaturated monomer comprises from about 30 to about 60 wt % of butyl acrylate.

21. The composite substrate of claim 1, wherein prior to polymerization, the at least one ethylenically unsaturated monomer comprises from about 50 to about 60 wt % of butyl acrylate.

22. The composite substrate of claim 1, wherein prior to polymerization, the at least one ethylenically unsaturated monomer comprises from about 10 to about 25 wt % of methyl methacrylate.

23. The composite substrate of claim 1, wherein prior to polymerization, the inverse phase photo-copolymerization comprises from about 1 to about 50 wt % of at least one ethylenically unsaturated monomer cross-linker.

24. The composite substrate of claim 1, wherein prior to polymerization, the inverse phase photo-copolymerization comprises from about 5 to about 30 wt % of at least one ethylenically unsaturated monomer cross-linker.

25. The composite substrate of claim 1, wherein prior to polymerization, the inverse phase photo-copolymerization comprises from about 10 to about 30 wt % of at least one ethylenically unsaturated monomer cross-linker.

26. The composite substrate of claim 1, wherein prior to polymerization, the inverse phase photo-copolymerization comprises from about 10 to about 20 wt % of at least one ethylenically unsaturated monomer cross-linker.

27. The composite substrate of claim 1, further comprising at least one non-reflective additive intercalated within the porous copolymer-monolith.

28. The composite substrate of claim 27, wherein the non-reflective additive is carbon black.

29.-38. (canceled)
